(19) United States
(12) Patent Application Publication
(10) Pub. No.: US 2016/0166329 A1
(43) Pub. Date: Jun. 16, 2016

(54) TOMOGRAPHIC IMAGING FOR INTERVENTIONAL TOOL GUIDANCE

(71) Applicant: GENERAL ELECTRIC COMPANY, SCHENECTADY, NY (US)

(72) Inventors: David Allen Langan, Clifton Park, NY (US); Bernhard Erich Hermann Claus, Niskayuna, NY (US); Omar Al Assad, Niskayuna, NY (US); Gregoire Avignon, New York, NY (US)

(21) Appl. No.: 14/570,988

(22) Filed: Dec. 15, 2014

(51) Int. Cl. A61B 19/00; A61B 6/00; A61B 6/02

(52) U.S. Cl. CPC A61B 19/5244; A61B 6/025; A61B 6/54; A61B 6/4014; A61B 2019/5238

(57) ABSTRACT

The present disclosure relates to the identification and tracking of a navigational instrument (e.g., a needle) in three-dimensions, with substantially real-time image updates of the instrument and updates of the tissue at an equal or lower rate. In certain embodiments, the images are acquired using a C-arm tomosynthesis system configured to move the X-ray source and detector in respective planes above and below the patient.

### BACKGROUND

The subject matter disclosed herein relates to imaging techniques for use in a navigational procedure, such as to provide guidance for insertion and navigation of a needle or other tool during a procedure.

Various medical procedures involve the insertion and navigation of a tool within a patient's body. For example, needle-based procedures (e.g., lung biopsy, vertebroplasty, RF ablation of liver tumors, and so forth) may involve the insertion and navigation of a needle or needle associated tool through the body of a patient. Such procedures are guided and, therefore, benefit from the acquisition and display of real-time imaging data to assist in the navigation process. For example, such image data may be used to safely guide the device to the target while avoiding critical structures (e.g., arteries and veins) and obstructions (e.g., bones).

Such image data may be acquired using various types of imaging modalities that employ various radiological principles. For example, technologies such as X-ray fluoroscopy, X-ray computed tomography (CT), and tomosynthesis use various physical principles, such as the varying transmission of X-rays through a target volume, to acquire projection data and to construct images (e.g., three-dimensional, volumetric representations of the interior of the human body or of other imaged structures).

The navigational imaging process, however, may be complicated by a variety of factors including, but not limited to: patient access (i.e., the spatial limitations imposed on one or both of the clinician inserting and guiding the device or the imaging system in the shared space), imaging artifacts resulting from the metal composition of the device, potential low contrast imaging challenges of the clinical target and surrounding structures, the ability to contend with temporal dynamics (e.g., motion, for example: quickly capturing the projections which are used to reconstruct the 3D volume so as to minimize motion effects), and the ability to rapidly acquire and reconstruct the data so as to enable the clinician to visualize tool and physiologic motion in 3D.

### BRIEF DESCRIPTION

In one embodiment, a navigational tracking method for visually tracking a navigational tool is provided. In accordance with this embodiment, a set of projection data is acquired data using an X-ray source and detector at a first frequency or when prompted by a user or physiological monitor. The set of projection data is acquired over a limited angular range by continuously moving the X-ray source and detector in an orbital trajectory with respect to a navigational volume such that the X-ray source and detector each stay on different respective sides of the navigational volume. A position of a high-contrast instrument is determined within the set of projection data or images generated using the set of projection data. An instrument representation of the high-contrast instrument is updated in the navigational volume at a first rate based on the determined position. A tissue representation depicting low-contrast structures within the navigational volume is updated at a second rate that is equal to or less than the first rate. The low-contrast structures include a target structure or region of the high-contrast instrument.

In an additional embodiment, a navigational imaging system is provided. In accordance with this embodiment, the navigational imaging system includes an X-ray source configured to move on a first side of a patient support and a detector configured to move on a second side of the patient support opposite the first side. The navigational imaging system also includes a controller and one or more processing components configured to, alone or in combination: continuously move the X-ray source and detector along orbital trajectories within a limited angular range and to operate the X-ray source and detector; acquire a set of projection data at a first frequency or when prompted by a user or physiological monitor; determine a position of a high-contrast instrument using the set of projection data or images generated using the set of projection data; update an instrument representation of the high-contrast instrument in a navigational volume at a first rate; and update a tissue representation depicting low-contrast structures within the navigational volume at a second rate that is equal to or less than the first rate. The low-contrast structures include a target structure or region of the high-contrast instrument.

In a further embodiment, a navigational tracking method is provided. In accordance with this embodiment, an initial volume comprising a target of a navigational procedure and surrounding tissue is generated. A representation of a navigational tool is depicted within the initial volume. A location of the navigational tool is determined using a subset of projections or images generated from the subset of projections derived from a larger set of projections. The representation of the navigational tool within the initial volume is updated, at a first rate, based on the location. The depiction of the target and surrounding tissue is updated, at a second rate, based on at least the larger set of projections.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical view of an imaging system for use in producing images in accordance with aspects of the present disclosure;

FIG. 2 is a schematic side view of a bi-plane imaging system in which a first imaging apparatus and a second imaging apparatus each obtain projection data along a plane, and the first imaging apparatus obtains projection data via rotation about two axes, in accordance with aspects of the present disclosure;

FIG. 3 depicts movement of a source and detector of a C-arm tomosynthesis system in accordance with aspects of the present disclosure;

FIG. 4 is a process flow diagram depicting steps of an algorithm for determining C-arm operating parameters, in accordance with aspects of the present disclosure; and

FIG. 5 is a process flow diagram depicting steps of an algorithm for generating navigational images, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

In certain navigational procedures, it is useful to be able to visualize the internal structures of a patient as part of the procedure. Aspects of the present approach utilize C-arm tomosynthesis to provide such guidance. As discussed herein, in certain embodiments, the X-ray detector and source (e.g., an X-ray tube) continuously orbit within a plane, respectively above and below the patient support table. In this arrangement, access to the patient is significantly improved relative to computed tomography (CT) imaging system or conventional Cone Beam Computed Tomography (CBCT) with a C-arm imaging system. The present approach also offers improved imaging and 3D resolution relative to conventional radiological approaches as well as a temporal image sampling rate or update rate that is sufficient for real-time (or near real-time) tracking in navigational procedures and superior to what is typically obtained in CBCT imaging.

As discussed herein, the present approach provides updated 3D imaging of the navigated device and of small, high-contrast structures for navigation. In particular, the needle (or other device or tool) is typically a high contrast structure which, along with other high contrast structures, can be identified and localized using relatively few images obtained at different view angles and this information may be used to update a separately or previously acquired volume or image depicting low-contrast structures, such as tissue (including the navigational target region, e.g., a tumor). Such an approach may be particularly useful for identifying and localizing a curved tool, such as a curved needle, whose curvature might be difficult to discern in two-dimensional or stereoscopic contexts. Large, low contrast structures may be best viewed in slices parallel to the orbit plane (generally coronal). In certain embodiments, the tool may be detected and tracked in the coronal slices and painted into the coronal slices, or into the 3D rendering. In another embodiment, lateral views may be utilized for reconstruction of the anatomy including the target region, thereby enhancing image quality through a more complete sampling of the volume. The lateral views may be acquired prior to the tomosynthesis acquisition or at the onset of the tomosynthesis acquisition. Further, fusion with pre-procedure 3D imaging, generally CT or CBCT imaged volumes, may be employed.

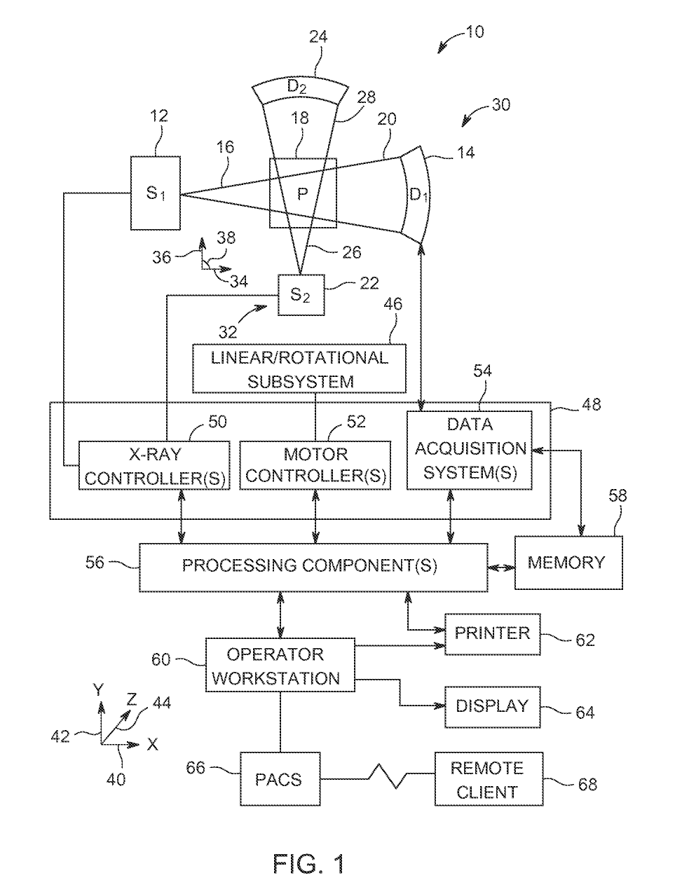

With the preceding in mind, an example of a bi-plane tomosynthesis imaging system 10 designed to acquire X-ray attenuation data at a variety of views around a patient and suitable for navigational imaging is provided in FIG. 1. In the embodiment illustrated in FIG. 1, imaging system 10 includes a first source of X-ray radiation 12 and a first detector 14. The first X-ray source 12 may be an X-ray tube, a distributed X-ray source (such as a solid-state or thermionic X-ray source) or any other source of X-ray radiation suitable for the acquisition of medical or other images. In certain implementations, the X-ray source 12 may be switchable between different emission profiles (e.g., profiles having different mean energy), such as to facilitate dual-energy imaging protocols.

The X-rays 16 generated by the first source 12 pass into a region in which a patient 18, is positioned during a procedure. In the depicted example, the X-rays 16 are collimated to be a cone-shaped beam, e.g., a cone-beam, which passes through the imaged volume. A portion of the X-ray radiation 20 passes through or around the patient 18 (or other subject of interest) and impacts a detector array, represented generally as the first detector 14. Detector elements of the first detector 14 produce electrical signals that represent the intensity of the incident X-rays 20. These signals are acquired and processed to reconstruct images of the features within the patient 18.

In the depicted example, the bi-plane imaging system 10 includes a second source 22 of X-ray radiation and a second detector 24, which, like the first detector 14, may include an array of detector elements. The second source 22 also generates X-rays 26, which may be collimated to form any suitable shape (e.g., a cone) and, in some instances may be switchable between different emission profiles. The X-rays 26 are partially attenuated such that a portion 28 passes through the patient 18 and impacts the second detector 24. The second detector 24 generates electrical signals, which are acquired and processed to reconstruct images of the features within the patient 18. Though the depicted tomosynthesis system 10 depicts two separate imaging subsystems (i.e., a first source and detector and a second source and detector), it should be understood that this illustration is merely for completeness and is only one example of a suitable system for implementing the present approaches. Indeed, the present approach may also be employed with a tomosynthesis system having only a single imaging subsystem (i.e., a single-plane system) or having more than two such imaging subsystems (i.e., bi-plane or multi-plane). A single plane system may be more widely available, and may offer benefits, e.g., in terms of superior patient access, while a bi-plane (or multi-plane) system may offer benefits, e.g., in terms of superior 3D image quality for soft-tissue imaging.

In the present example, the first source 12 and first detector 14 may be a part of a first imager 30. The first imager 30 may acquire X-ray images or X-ray projection data over a limited angular range with respect to one side or facing (e.g., the anterior/posterior (AP) direction) of the patient 18, thereby defining data in a first plane (e.g., a frontal plane of the patient 18). The second source 22 and the second detector 24, if present and employed, may be a part of a second imager 32. The second imager 32 may acquire data within a different limited angular range with respect to a different side or facing (e.g., a lateral direction) of the patient 18, thereby defining data in a second plane (e.g., a lateral plane of the patient 18). In this context, an imaging plane may be defined as a set of projection directions that are located within a certain angular range relative to a reference direction. For example, the frontal imaging plane may be used to describe projection views within an angular range that is within, for example, 60 degrees of the PA (posterior/anterior) direction of the patient. Similarly, the lateral imaging plane may be described as the set of projection directions within an angular range that is within 60 degrees of the lateral/horizontal left/right projection direction. A variety of configurations may be employed where the first and second imagers 30, 32 obtain data that may be jointly used, such as to construct and/or update one or more three-dimensional images of the patient 18 (e.g., tissues of interest of the patient 18).

As depicted, the first imager 30 positions the first source 12 and the first detector 14, at rest, generally along a first direction 34, which may correspond to the AP direction of the patient 18 in certain embodiments. The second imager 32 positions the second source 22 and the second detector 24, at rest, generally along a second direction 36, which may correspond to the lateral direction of the patient 18 in certain embodiments. The first and second directions 34, 36 may be oriented at an angle 38 relative to one another. The angle 38 may be any angle that is suitable to enable the first and second imagers 30, 32 to acquire projection data over separate and distinct limited angular ranges with respect to the patient. Further, the angle 38 may be adjusted by various features of the system 10, such as various linear and rotational systems or, in other embodiments, by an operator. Generally, the angle 38 may be between 30 and 180 degrees, but it may be desirable in certain embodiments for the first and second imagers 30, 32 to be oriented crosswise relative to one another, such as between 30 and 90 degrees, or between 90 and 150 degrees. In one embodiment, the angle 38 is approximately 90 degrees.

In accordance with present embodiments, the first imager and the second imager 30, 32 may be moved relative to the patient or imaged object and relative to one another along one or more axes during an examination procedure during which projection data is acquired. For example, the first imager 30 may move about a first axis of rotation 40, a second axis of rotation 42, or a third axis of rotation 44, or any combination thereof, and the second imager 32 may move about any one or a combination of these axes as well. Each imager may have its own independent axes of motion (e.g., translation and rotation), or some axes may be shared. In one embodiment, the rotation of the first and second imagers 30, 32 may be coordinated in accordance with a specified protocol. In a further implementation, the second imager 32 may be stationary and may, therefore, only acquire projection data from a fixed position relative to the imaged object.

The movement of the first imager 30 and/or the second imager 32 may be initiated and/or controlled by one or more linear/rotational subsystems 46. The linear/rotational subsystems 46, as discussed in further detail below, may include support structures, motors, gears, bearings, and the like, that enable the rotational and/or translational movement of the imagers 30, 32. Additionally, the linear/rotational subsystems 46 may include positional encoder devices to measure the motion about an axis and communicate the measurement back to the motor control 52. In one embodiment, the linear/rotational subsystems 46 may include a first structural apparatus (e.g., a C-arm apparatus having rotational movement about at least two axes) supporting the first source and detector 12, 14, and a second structural apparatus (e.g., a C-arm apparatus) supporting the second source and detector 22, 24. In another embodiment, support structures and/or linear/rotational subsystems for the source and detector in at least one imager are independent, in which case the source and detector of that imager may each have their own support and positioning subsystems.

A system controller 48 may govern the linear/rotational subsystems 46 that initiate and/or control the movement of the first and second imagers 30, 32. In practice, the system controller 48 may incorporate one or more processing devices that include or communicate with tangible, non-transitory, machine readable media collectively storing instructions executable by the one or more processors to perform the operations described herein. The system controller 48 may also include features that control the timing of the activation of the first and second sources 12, 22, for example, to control the acquisition of X-ray attenuation data obtained during a particular imaging sequence. The system controller 48 may also execute various signal processing and filtration functions, such as for initial adjustment of dynamic ranges, interleaving of digital projection data, and so forth. Therefore, in general, the system controller 48 may be considered to command operation of the imaging system 10 to execute examination protocols. It should be noted that, to facilitate discussion, reference is made below to the system controller 48 as being the unit that controls acquisitions, movements, and so forth, using the imagers. However, embodiments where the system controller 48 acts in conjunction with other control devices (e.g., other control circuitry local to the imagers or remote to the system 10) are also encompassed by the present disclosure.

In the present context, the system controller 48 includes signal processing circuitry and various other circuitry that enables the system controller 48 to control the operation of the first and second imagers 30, 32 and the linear/rotational subsystems 46. In the illustrated embodiment, the circuitry may include an X-ray controller 50 configured to operate the first and second X-ray sources 12 and 22 so as to time the operations of these sources and to interleave the acquisition of X-ray attenuation data when needed. Circuitry of the system controller 48 may also include one or more motor controllers 52. The motor controllers 52 may control the activation of various components that are responsible for moving the first and second sources 12, 22 and the first and second detectors 14, 24. For example, the motor controllers 52 may coordinate movement of the first and second imagers 30, 32 such that the imagers obtain data from different projection directions, maintain a desired degree of angular separation, and also for collision avoidance. In other words, the motor controllers may implement a particular trajectory for one or both of the first and second imagers 30, 32.

The system controller 48 is also illustrated as including one or more data acquisition systems 54. Generally, the first and second detectors 14, 24 may be coupled to the system controller 48, and more particularly to the data acquisition systems 54. The data acquisition systems 54 may receive data collected by read out electronics of the first and second detectors 14, 24, and in certain embodiments may process the data (e.g., by converting analog to digital signals or to perform other filtering, transformation, or similar operations).

It should be noted that the tangible, non-transitory, machine-readable media and the processors that are configured to perform the instructions stored on this media that are present in the system 10 may be shared between the various components of the system controller 48 or other components of the system 10. For instance, as illustrated, the X-ray controller 50, the motor controller 52, and the data acquisition systems 54 may share one or more processing components 56 that are each specifically configured to cooperate with one or more memory devices 58 storing instructions that, when executed by the processing components 56, perform the image acquisition techniques described herein. Further, the processing components 56 and the memory components 58 may coordinate in order to perform various image reconstruction processes.

The system controller 48 and the various circuitry that it includes, as well as the processing and memory components 56, 58, may be accessed or otherwise controlled by an operator via an operator workstation 60. The operator workstation 60 may include any application-specific or general-purpose computer that may include one or more programs (for example one or more imaging programs) capable of enabling operator input for the techniques described herein. The operator workstation 60 may include various input devices such as a mouse, a keyboard, a trackball, a touchscreen, or any other similar feature that enables the operator to interact with the computer. The operator workstation 60 may enable the operator to control various imaging parameters, for example, by adjusting certain instructions stored on the memory devices 58. In one embodiment the operator workstation may enable the operator to perform one or more of marking the target region in 3D (e.g., by tracing an outline of a tumor), (partially) planning the trajectory of a device (e.g., by tracing the intended path of a needle), choosing a center view around which the tomosynthesis acquisition will be centered, or setting up other acquisition parameters, and so forth.

The operator workstation 60 may be communicatively coupled to a printer 62 for printing images, patient data, and the like. The operator workstation 60 may also be in communication with a display 64 that enables the operator to view various parameters in real time, to view images produced by the acquired data, and the like. The operator workstation 60 may also, in certain embodiments, be communicatively coupled to a picture archiving and communication system (PACS) 66. Such a system may enable the storage of patient data, patient images, image acquisition parameters, and the like. This stored information may be shared throughout the imaging facility and may also be shared with other facilities, for example, a remote client 68. The remote client 68 may include hospitals, doctors' offices, or any other similar client.

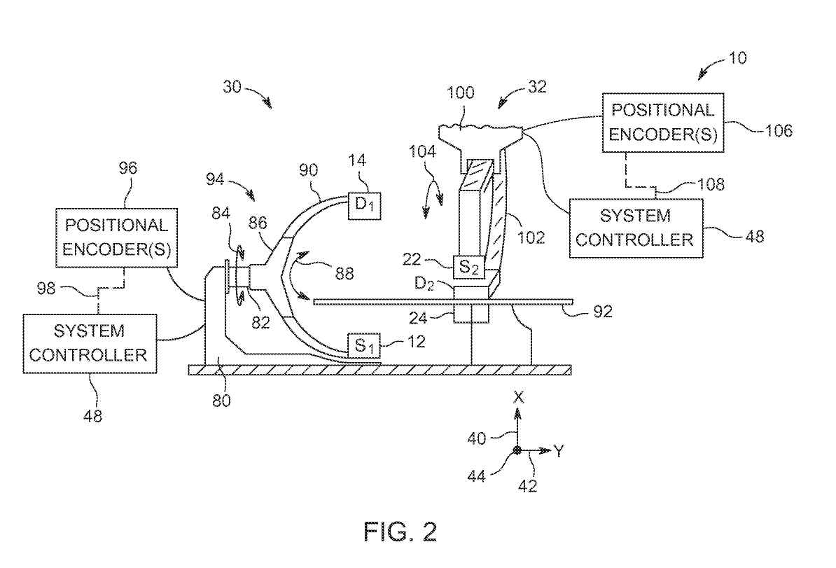

Various aspects of the present approaches may be further appreciated with respect to FIG. 2, which is a side view of an embodiment of the system 10. As illustrated, the system 10 includes the first imager 30 and the second imager 32. It should be noted that in practice, the second imager 32 may actually be closer in space to the first imager 30 than as illustrated in FIG. 2 (e.g., moved to the left in the illustration). However, to facilitate discussion of the present techniques and for clarity, the second imager 32 is depicted as being positioned further away from where it would, in practice, image the patient 18. The first imager 30, as illustrated, includes a first base 80 and a rotatable extension 82 extending from the first base 80. In the illustrated embodiment, the first base 80 is a floor-mounted base such that the first imager 30 may be secured to a floor of an imaging area in which it is positioned. In other embodiments, however, the first base 80 may not be secured to the floor (e.g., may be movable, or may be mounted to the ceiling, etc.).

The rotatable extension 82 is depicted as extending generally along the second axis of rotation 42, and enables the first source 12 and the first detector 14 to move about the second axis of rotation 42. For example, the rotatable extension 82 may enable the first source 12 and the first detector 14 to move about the second axis of rotation 42 in a manner that maintains their position relative to one another throughout the rotation. The rotation enabled by the rotatable extension 82 is shown as double-headed arrow 84. The rotatable extension 82 is coupled to a first moving structure 86 (e.g., directly or indirectly via an extension arm), which enables the first source 12 and the first detector 14 to move about the third axis of rotation 44. This rotation about the third axis of rotation 44 is depicted as double-headed arrow 88.

The first moving structure 86 may be a geared or track structure that is motively coupled to a first support structure 90 that physically supports the first source 12 and the first detector 14, and may be in the form of a C-arm, or any other shape that positions the first source 12 and the first detector 14 on either side of the patient 18. As illustrated, the first support structure 90 includes an arcuate structure that extends from a first side of a patient table 92, around the patient table 92, and to a second side of the patient table 92. In this way, the first source 12 and the first detector 14 generally remain positioned at opposite ends and/or on opposite sides of the patient (not shown) positioned on patient table 92. Together, the first base 80, the rotatable extension 82, the first moving structure 86, and the first support structure 90 may be considered to be the first structure 94 of the first imager 30.

The first imager 30 may include various motors, actuators, or other features responsible for movement of the various structures of the first imager 30, and they may be communicatively coupled to one or more positional encoders 96. One or more positional encoders 96 may encode the respective positions of any one or more components of the first imager 30 in a manner that facilitates processing by the system controller 48. In such an implementation, the positional encoders 96 may provide feedback 98 (for example via wired or wireless signals) to the system controller 48. The system controller 48 may use this feedback 98 to control either or both the first imager 30 and the second imager 32.

In the illustrated embodiment, the second imager 32 is depicted as including a second base 100. The second base 100 may be mounted to any structure, or may be a mobile base. However, in the illustrated embodiment, the second base 100 is depicted as a ceiling-mounted structure. The second base 100 may also include various motive devices such as gears, actuators, tracks, or any similar features that enable movement of the second source 22 and the second detector 24. Specifically, the second base 100 is physically and motively coupled to a second support structure 102, which is depicted as a curved structure that suspends the second source 22 and the second detector 24 on opposite sides or ends of the patient table 92 (e.g., along a lateral direction of the patient 18). The motive devices or similar features of the second imager 32 may operate to move the second source 22 and the second detector 24 about the patient table 92 in one or more rotational directions.

In one embodiment, the second source 22 and the second detector 24 may move about the second axis of rotation 42 or another axis of rotation. The rotation by the second imager 32 is depicted as double-headed arrow 104. In some embodiments, the data acquired with the second imager 32 is used as a partial set of data that is used to reconstruct a 3D volume. Obtaining data along the additional trajectory traversed by the second imager 32 may be desirable to obtain data that can be useful in reconstructing three-dimensional images from incomplete data sets acquired using the first imager 30.

Like the first imager 30, the second imager 32 is depicted as being communicatively coupled (for example via wired or wireless communication) to one or more positional encoders 106, which may be shared with the first imager 30 or may be entirely separate from the first imager 30. The positional encoders 106 may encode the position of any one or more of the second base 100, the second support structure 102, the second source 22 or the second detector 24, or any other feature of the second imager 32. The positional encoder 106 may provide feedback 108 to the system controller 48 to enable the system controller 48 to determine the position of the features of the second imager 32 relative to the features of the first imager 30, or relative to any other appropriate reference (e.g., a three-dimensional space established by one or more devices that provide and/or control position information of system components and/or devices).

As an example, the system controller 48 may simultaneously move the first source 12 and the first detector 14 together about the first axis of rotation 40, the second axis of rotation 42, or the third axis of rotation 44, or any combination thereof, and obtain first X-ray attenuation data for a subset of the traversed view angles. At substantially the same time, the system controller 48 may simultaneously move the second source 22 and the second detector 24 together about the first, second, or third axes of rotation 40, 42, 44, or any combination thereof, in order to obtain second X-ray attenuation data for one or more of the traversed view angles. In one embodiment, the system controller 48 may receive positional information from the positional encoders 96, 106, relating to the first imager 30 and the second imager 32, and may calculate a trajectory (or update a modeled trajectory) for either or for both of the first source and detector 12, 14 and the second source and detector 22, 24 using this positional feedback information.

Furthermore, the system controller 48 may synthesize one or more volumetric images using data obtained by the first imager 30. In one embodiment, the angular range of the trajectory may be limited due to the presence of other devices or structures, or due to temporal constraints. In one example, the angular range of an elliptical orbit that is part of the trajectory may be defined by the requirement that the orbit may have to be traversed in a certain amount of time, e.g., in 3 seconds or less. For continuous real-time tracking, such an orbit may be traversed multiple times while the device (e.g., needle) is being navigated towards the target region, thereby continuously updating image and positional information about the device. Various tomosynthesis reconstruction algorithms that may be used to reconstruct a 3D volumetric image of the imaged region of interest include those that are well known by those of ordinary skill in the art, and may be of the analytical or iterative type, including but not limited to filtered back projection.

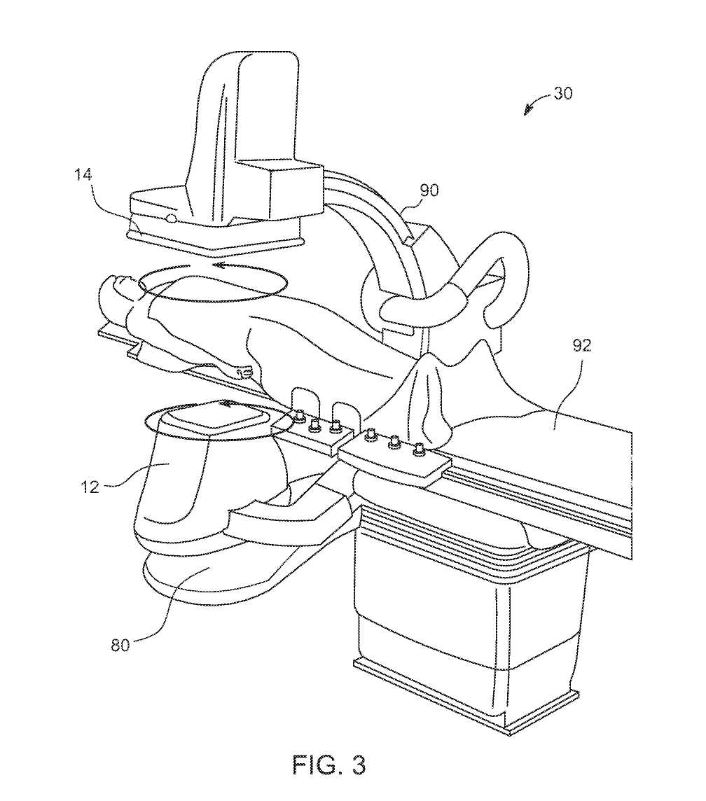

In some instances, the system controller 48 may synthesize one or more volumetric images using data obtained by the first imager 30 supplemented by data obtained by the second imager 32. For example, in one embodiment, projection images/data obtained by the second imager 32 may be used to supplement the data obtained by the first imager 30, such as for reconstruction of a 3D image. In such an embodiment, the first imager 30 may perform a first acquisition of data using a first trajectory (e.g., a circular, ellipsoidal, or similar path traced by the first source 12 below the patient 18 and a corresponding circular, ellipsoidal, or similar path traced by the first detector above the patient 18, referred to herein as a frontal tomosynthesis trajectory). An example of a section of such a motion trajectory (i.e., an “orbit” as used herein) is conceptually demonstrated in FIG. 3 in the context of a first imager 30. In this example, the first imager 30 may obtain projection data from a plurality of projection directions, but these projection directions may also be limited by the angular range of motion of the first imager 30 (e.g., the limited angular displacement about the second rotational axis 42) and/or the presence of structures associated with the second imager 32, or other devices or structures.

In accordance with certain embodiments, a second imager 32 may move about the second rotational axis 42 at projection directions beyond those obtained by the first imager 30 (e.g., at larger angles relative to the frontal plane of the patient 18). Thus, the data obtained by a second imager 32, if present, may complement the data obtained by the first imager 30, and may enable the system controller 48 (or other reconstruction device) to perform 3D tomosynthesis reconstruction using a more complete set of data. This data may be considered to be obtained by the second imager 32 via lateral plane imaging, in that the second X-ray source 22 may generate a trajectory that may trace a line or non-linear path along a lateral direction of the patient 18 (and at angular displacements therefrom). In certain embodiments, data acquisition by the first and second imagers 30, 32 may be interleaved in order to avoid signal contamination between the imagers. In one embodiment, in a system with a single imager, the imager may be moved (intermittently, and/or before the start of the navigational procedure, e.g., for a planning acquisition) into a lateral position in order to acquire data in a lateral orientation, and may be moved back to the frontal position for imaging during the navigational procedure.

With the preceding in mind, as used herein, a tomosynthesis trajectory of an imager may be described as a path (e.g., a line, curve, circle, oval, and so forth, as well as combinations thereof) traced by an X-ray source during image acquisition. A tomosynthesis acquisition by an imager or imager subsystem occurs over a limited angular range with respect to the patient (such as with respect to one side, e.g., the front, back, left side, or right side, of the patient), and thus a trajectory will typically move the source within this limited angular range with respect to the imaged subject. Such trajectories may be periodic in that the path traced by the X-ray source may be repeated throughout the examination. In some instances, the trajectory may be adapted throughout the process, e.g., in order to accommodate patient access, optimize the view angle for visualization of anatomical structures, and so forth. In one embodiment, the tomographic angle for the trajectory is increased periodically (e.g., periodically the gantry will traverse a larger ellipse or similar trajectory), in order to maximize image quality for visualization of the target region (e.g., tumor), while at other times the trajectory may consist of smaller ellipses that enable real-time tracking of the device.

As noted above, and as shown in FIG. 3, each period of motion may be referred to as an orbit. For example, in the context of an oval or circular trajectory, an endpoint of one orbit may correspond to the beginning point of the next orbit. Similarly, linear or non-linear paths traced by the X-ray source may be repeated in a back-and-forth manner, leading to a periodic type trajectory. For example, an X-ray source may be moved (i.e., have a trajectory) in a circular or oval periodic motion (e.g., an orbit) in front of the patient, without rotating around the patient, thereby acquiring X-ray projection data over a limited angular range with respect to the patient. By way of example, the present approach relates to the use of a C-arm to perform tomosynthesis imaging in a navigational context. In this imaging mode, the detector 14 and tube (e.g., source 12) orbit continuously within a plane above and below the table 92. In one embodiment, the orbit generally has a half tomographic angle of 15° to 30° (i.e., the view directions, as defined by a line connecting the source point with the center of the detector, are within a 15 to 30 degree angular range from a central view direction throughout the tomosynthesis trajectory) and an orbit period of 3 to 8 seconds. Such a motion is in contrast to the spin-type source motion or trajectory typically associated with computed tomography (CT) type systems and acquisitions.

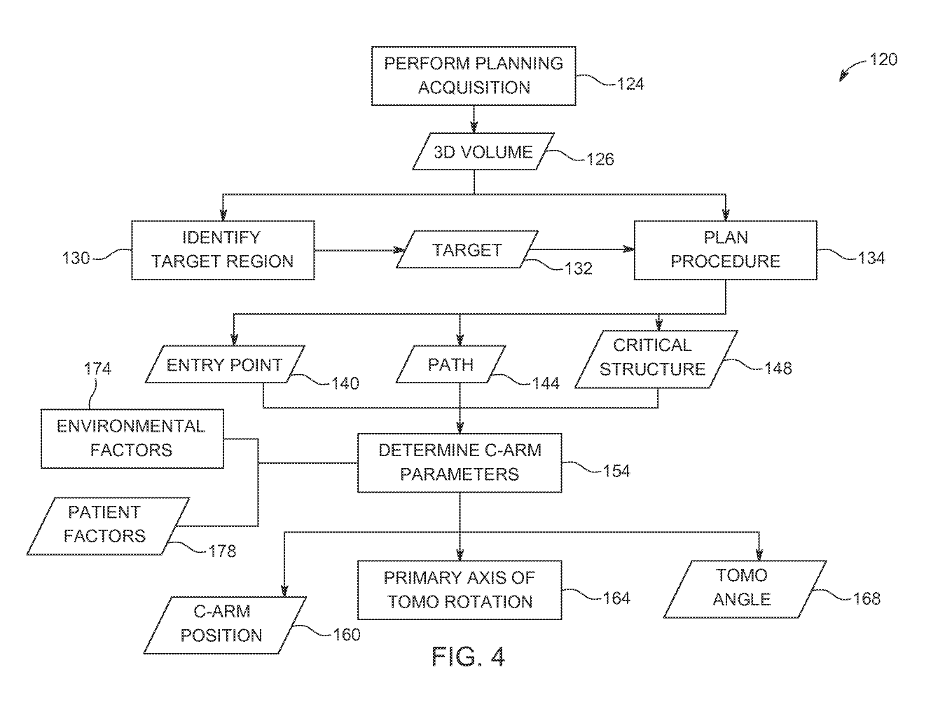

With the preceding in mind, and turning to FIG. 4, a process flow 120 is depicted showing steps of one such embodiment. In this example, prior to needle insertion and navigation, an initial planning acquisition (e.g., a CBCT or CT image acquisition) may be performed (block 124) to generate a three-dimensional (3D) volume 126 of the anatomical area of the patient where the procedure is to be performed. While the planning acquisition may be performed using a CT or CBCT modality in certain embodiments, in other embodiments the planning acquisition may be a large-angle single-plane, or bi-plane tomosynthesis acquisition, such as may be acquired using second source 22 and second detector 24 of a bi-plane tomosynthesis system.

The 3D volume 126 may be used for planning a navigational procedure. For example, the 3D volume 126 may be used to identify (block 130) the target structure or region 132 and to plan the procedure (block 134), such as by determining an entry point 140, a path 144, and identification of critical structures 148 to avoid, and so forth. The procedure plan (e.g., target 132, entry point 140, path 144, and structures to avoid 148) may be used to determine (block 154) operating parameters under which the primary C-arm to be moved during navigation of the tool will operate during the procedure. Examples of such C-arm operating parameters may include, but are not limited to C-arm position 160 (e.g., L-arm position), primary axis of rotation 164, and the tomosynthesis angle 168. For example, the primary axis of rotation may be selected such that the path of the device, as well as the target region, are approximately located within a plane that is parallel to the plane traversed by the tube (or the primary beamline) during the tomosynthesis acquisition.

In addition, other factors may be taken into account in determining the C-arm operating parameters. For example, patient factors 174, such as the position and size of the patient may be considered in determining the C-arm operating parameters. Similarly, environmental factors or considerations 178, such as room layout, obstructions in the vicinity of the patient (e.g., anticipated position of clinician, anesthesia cart, and so forth) may be taken into account in determining the C-arm operating parameters. Similarly, dose consideration may be taken into account so as to minimize patient X-ray dose. Such dose considerations may affect selection of the tomosynthesis axis, the tomosynthesis angle, and/or the collimator setting. In one embodiment, the collimator settings may be updated during parts of the acquisition such that the X-ray beam is collimated to a small region that may encompass the tip of the device/needle and/or the target region. At other times the collimator may be in the fully open position such as to enable imaging of the target region, the tool, and the anatomical context.

By way of example, an initial volumetric dataset 126 may be acquired with a large-angle tomosynthesis acquisition. This initial dataset 126 may be used for identification of the target region 132, which may be selected and marked by an operator, as well as planning of the trajectory of the needle/tool, and so forth. As a function of the anatomy, patient access for insertion and navigation of the needle, and so forth, a primary tomosynthesis axis and tomographic angle is selected, either automatically or manually.

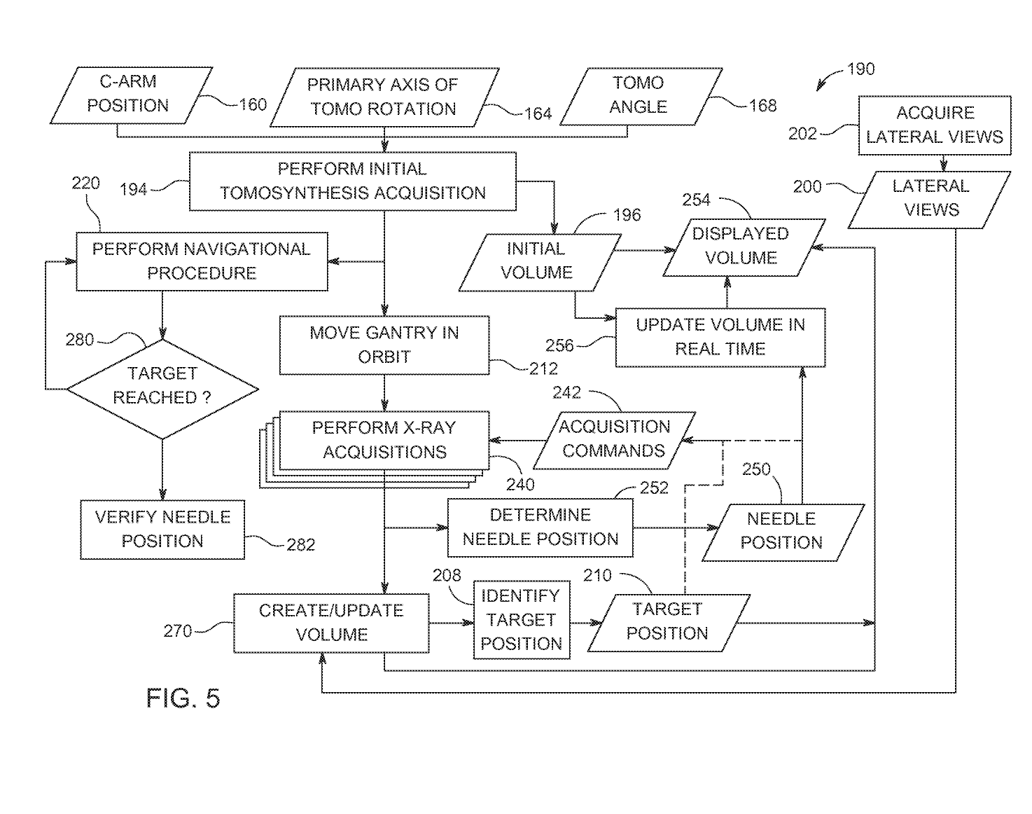

Turning to FIG. 5, once the C-arm operating parameters are determined, a navigational procedure with accompanying imaging may be performed (block 220), as shown in process flow 190. In the depicted example, an initial tomosynthesis acquisition (block 194) of the patient is performed producing an initial volume 196. In some implementations pre-procedure lateral views 200 are also acquired (block 202) and a reconstructed volume formed (block 270) using projection data from both, primary and lateral beamline, may be used in the identification (block 208) of the target position 210. In certain embodiments, one or both of the tomosynthesis acquisition or the lateral acquisition may be performed using a dual-energy protocol, which may allow generation of soft tissue images, bone images, or material decomposition images.

In one embodiment, the tomosynthesis gantry continuously orbits (block 212) during the needle (or other trackable tool) insertion and guidance. In certain implementations, gantry speed and/or trajectory may be varied (such as between small orbits used to obtain navigational projection data and larger orbits used to obtain tissue projection data) or the gantry may be intermittently stopped, such as under the control of a physician involved in the navigational procedure, to provide temporary access to the patient, such as to the needle insertion site. X-ray acquisitions (block 240) (e.g., X-ray exposure and readout events) may be gated or otherwise controlled in response to commands or prompts 242 provided by an operator, such as via a foot pedal or other device in control of the operator. X-ray frame rate, as well as other acquisition parameters may be similarly adapted or varied during the course of the procedure. In practice X-ray acquisitions may occur over an extended time frame (e.g., over the course of the navigational procedure) and may contribute to multiple image volumes over that extended time frame. In addition, in some implementations, physiological gating may be employed in addition to or instead of operator-initiated acquisitions. Further, as shown in FIG. 5, acquisitions 242 may be prompted in response to or taking in to account target position 210 and/or needle position 250 over the course of the procedure. For example, based on the current target or needle position and/or orientation, or on changes to the target or needle position, additional acquisitions 242 may be prompted. Likewise, acquisitions may be prompted based on an elapsed time since a previous acquisition, or an elapsed time since a needle or target position update. As with the initial images, tomosynthesis acquisitions 240 during the procedure may also be performed in accordance with a dual-energy imaging protocol so as to facilitate generation of soft-tissue, bone, or material decomposition images.

Because, the needle or other navigated tool is typically a small, high contrast, well-defined object, 3D position 250 may be determined (block 252) from the acquired projections in substantially real-time and used to update (block 256) the initial volume 196 to depict the current position 250 of the needle in an updated or displayed volume 254. In one embodiment, the update of the volume may consist of in-painting the estimated location of the tool/needle. In certain embodiments, the needle and surrounding tissue may be visualized in three-dimensions (i.e., as a volumetric representation) to convey the position, orientation, and curvature of the needle (or other tool) in the context of the surrounding tissue. In another embodiment, the information may be presented to the clinician in two-dimensions (2D), where an appropriate projection angle is selected and employed for the visualization so as to minimize any ambiguity in directing the needle to the target. In one embodiment, a “coronal” slice through the anatomy is displayed (i.e., a planar cross-section through the volume along a plane that is approximately parallel to the plane containing the trajectory of the source), where the plane is automatically selected such that it contains the tool and the target region. In one embodiment, multiple representations of the anatomy and/or the device are displayed concurrently, e.g., with additional markers (e.g., crosshairs) etc. in order to establish a spatial relationship between the representations.

In certain embodiments, the position 250 of the needle may be derived from a dynamically updated 3D reconstruction of a volume of interest (generated using the projections acquired at step 240) (i.e., based on a volume reconstruction) or based on a triangulation approach based on limited projection data and leveraging the high-contrast (and thus readily discernible) nature of the needle (or other device). For example, in one implementation of such a triangulation approach, the needle or other tool may be segmented (or pre-segmented) in the projection data (acquired at step 240) and the segmented data used to update a 3D model of the needle position, and so forth.

In one implementation, the update step 256 may utilize temporal smoothness constraints (e.g., Kalman filter type processing), which may be useful in detecting patient motion, modeling respiratory motion (i.e., periodic slight deformation of the imaged volume), and associated motion of the needle or other imaged tool. In addition, adaptive refinement of the imaging geometry may be employed during three-dimensional image processing to robustly handle an arbitrary axis of tomosynthesis rotation. When the clinician believes the needle has reached its target (decision block 280), CBCT or tomosynthesis with lateral views may be performed, if desired, to achieve higher image quality to verify (block 282) needle position 250.

With this in mind, as part of a “navigational mode” of the process 190 during which the emphasis is on acquiring and updating position information related to the needle or device, in one implementation the system orbits through an ellipse with an appropriate small diameter (e.g., a small elliptical orbit), at which time X-ray images are acquired with a slow frame-rate (or, in groups of 2 or more X-ray frames where the spacing between frames within a group is, for example, approximately 100-300 milliseconds, while spacing between groups may be of the order of 1.5 or more seconds). During this time (i.e., the “navigation mode”), the X-ray beam may be collimated down to encompass only the needle tip and/or the target region. During this step, the current needle/tool position is displayed on the most recent display volume that contains anatomical context and detail, as well as the target region.

While the needle or tool position may be updated near continuously or with great frequency as X-ray acquisitions 240 occur over time, in certain embodiments the target position 210 is updated (block 270) at a lower rate, such as at a rate consistent with procedural need. In particular, the target may generally be assumed to be low-contrast, therefore requiring a greater number of projections to visualize or detect than the tool (e.g., needle). Alternatively, in certain circumstances, such as where tissue deformation occurs in response to advancement of the tool, it may be useful to update the target position (or the overall tissue context) at the same rate as the needle, assuming sufficient projection data is available. In such an embodiment, the target position may be updated, for example, based on operator feedback, or based on registration with an earlier volume dataset.

With this in mind, as part of an “evaluation mode” of the process 190 during which the emphasis is on reconstruction of needle and its current position relative to the target region, as well as anatomical context and detail, and allows for evaluation of the progress of the procedure. In one implementation, at regular intervals, or as requested by the operator, X-ray data is acquired at a high frame rate. During this time (i.e., the “evaluation mode”), the gantry moves through a tomosynthesis trajectory with a larger angle (i.e., a larger or wider orbit). In one embodiment, the full field of view is imaged during the evaluation mode (i.e., the X-ray beam is not collimated down to a small area). The reconstructed volume may contain added special emphasis on the current needle position (e.g., in-painted), as well as the target region (e.g., as overlayed contour, or marker). In one embodiment, the volume may be reconstructed using reconstruction methods that employ strategies for metal artifact reduction that are known in the art. As the needle or other tool approaches the target, the high frame rate acquisition (i.e., the evaluation mode acquisition) may be repeated more often, in order to evaluate placement of the needle, potential deformation of the anatomy, and associated displacement of the target region. The location of the target region within the volume may be updated, either by registration, or by the operator. In the final stages of the procedure, a further acquisition in “evaluation mode” may be performed to verify placement of the needle tip relative to the target region. This final acquisition may also be performed using an even larger tomographic angle, or using other modified imaging parameters that enable improved image quality.

In one embodiment, acquisitions in the evaluation mode and the navigation mode are performed alternatingly. In one embodiment, there is a smooth transition of the gantry motion when switching between the two modes. During this transition X-ray images may be acquired, e.g., with a low frame rate. It should be appreciated that, though a navigational mode and an evaluation mode are specifically called out, the process 190 may also include other modes of operating the respective tomosynthesis imaging system during a navigational procedure. For example, a correction or adjustment mode may be provided during which no X-ray projections are acquired and instead adjustments or corrections to the procedure may be implemented. For example, during such a “dark” mode (i.e., no X-ray emissions), collision testing may be performed with respect to the moving imager components (e.g., in a bi-plane acquisition operation), on-the-fly adjustments may be made to gantry trajectories, and so forth.

In certain embodiments, a dynamic iterative reconstruction (or update to reconstruction) may be applied as part of the reconstruction process used to generate images from which the target tissue is identified and/or localized for the procedure 190. As shown in the depicted example, lateral views 200 obtained before or during the procedure may be incorporated into the target imaging and reconstruction, if appropriate, to supplement the image data acquired by the tomosynthesis acquisition process 240. Target localization may also be aided by registration of the current tomosynthesis data with prior imaging (e.g., CT or CBCT) data, and/or potentially with manual support. As may be appreciated, in the context of a navigational procedure and the accompanying image acquisition, patient motion is likely to occur to some extent. With this in mind, in certain implementations patient motion may be detected as a large scale registration error and corrected.

With the preceding discussion in mind, the following example is provided so as to illustrate one possible implementation in a concise and readily accessible form. In this example, an initial volumetric dataset may be acquired with a large-angle tomosynthesis acquisition. This initial display volume may be used for identification of the target region, which may be selected and marked by an operator, as well as planning of the trajectory of the needle/tool, and so forth. As a function of the anatomy, patient access for insertion and navigation of the needle, and so forth, a primary tomosynthesis axis and tomographic angle is selected, either automatically or manually. During the navigation of the needle the imager orbits through an ellipse with an appropriate small diameter, at which time X-ray images are acquired with a slow frame-rate (or, in groups of 2 or more X-ray frames where the spacing between frames within a group is, for example, approximately 100-300 milliseconds, while spacing between groups may be of the order of 1.5 or more seconds). During this time (i.e., the “navigation mode”), the X-ray beam may be collimated down to encompass only the needle tip and/or the target region. During this step, the current needle/tool position is displayed on the most recent display volume that contains anatomical context and detail, as well as the target region. In regular intervals, or as requested by the operator, X-ray data is acquired at a high frame rate. During this time (i.e., the “evaluation mode”), the gantry moves through a tomosynthesis trajectory with a larger angle. This acquisition allows for reconstruction of needle and target region, as well as anatomical context and detail, and allows for evaluation of the progress of the procedure. The reconstructed volume may contain the current needle position (e.g., in-painted), as well as the target region (e.g., as overlayed contour, or marker). When approaching the target region, this high frame rate acquisition may be repeated more often, in order to evaluate placement of the needle, potential deformation of the anatomy, and associated displacement of the target region. The location of the target region within the volume may be updated, either by registration, or by the operator. In the final stages of the procedure, another acquisition in “evaluation mode” may be performed to verify placement of the needle tip relative to the target region. This final acquisition may also be performed using an even larger tomographic angle, or using other modified imaging parameters that enable improved image quality.

As will also be appreciated, depending on the navigational procedure, it may be appropriate and/or necessary to occasionally administer a contrast bolus to facilitate visualization of the vasculature. Thus, contrast imaging procedures and other imaging procedures, such as breath holds, and so forth, may be incorporated as appropriate with respect to the present imaging approaches. It will also be appreciated that the devices/needles as discussed herein may be navigated/inserted percutaneously (e.g., a biopsy needle), as well as intravascular navigated devices (e.g., catheters for ablations, embolizations, etc.).

With the preceding in mind, technical effects of the invention include identifying and tracking a navigational instrument (e.g., a needle) in three-dimensions, with substantially real-time image updates of the instrument and updates of the tissue at a lower rate. Technical effects also include parameterizing a C-arm imaging trajectory based on an initially acquired volumetric image and one or both of environmental or patient specific factors. Technical effects also include acquiring 3D images of a tracked navigational instrument so as to be able to discern curvature of the navigational instrument within a 3D tracking representation.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A navigational tracking method for visually tracking a navigational tool, comprising:
acquiring a set of projection data using an X-ray source and detector at a first frequency or when prompted by a user or physiological monitor, wherein the set of projection data is acquired over a limited angular range by continuously moving the X-ray source and detector in an orbital trajectory with respect to a navigational volume such that the X-ray source and detector each stay on different respective sides of the navigational volume;
determining a position of a high-contrast instrument within the set of projection data or images generated using the set of projection data;
updating an instrument representation of the high-contrast instrument in the navigational volume at a first rate based on the determined position; and
updating a tissue representation depicting low-contrast structures within the navigational volume at a second rate that is equal to or less than the first rate, wherein the low-contrast structures include a target structure or region of the high-contrast instrument.

2. The navigational tracking method of claim 1, wherein the acquisition of the set of projection data transitions between a first mode and a second mode having different respective acquisition parameters, wherein the first mode is associated with the step of updating the instrument representation and the second mode is associated with updating the tissue representation.

3. The navigational tracking method of claim 2, wherein the respective acquisition parameters associated with the second mode comprise one or more of high frame rate, large tomographic angle, full field of view, high dose, or bi-plane acquisition.

4. The navigational tracking method of claim 2, wherein the respective acquisition parameters associated with the first mode comprise one or more of low frame rate, small tomographic angle, collimated views, or low dose.

5. The navigational tracking method of claim 2, wherein the act of updating the instrument representation uses only the projection data acquired in the first mode or projection data acquired in both the first mode and the second mode.

6. The navigational tracking method of claim 2, wherein the act of updating the tissue representation uses only the projection data acquired in the second mode or projection data acquired in both the first mode and the second mode.

7. The navigational tracking method of claim 2, further comprising applying a temporal smoothness constraint to one or both of the position or orientation of the high-contrast instrument.

8. The navigational tracking method of claim 2, wherein the respective acquisition parameters of one or both of the first mode or the second mode are adapted during a tracking operation based on a current position of the high-contrast instrument, an anatomical feature proximate to a tip of the high-contrast instrument, outputs of a physiological monitor, or operator inputs.

9. The navigational tracking method of claim 2, further comprising a third mode during which no X-ray projections are acquired.

10. The navigational tracking method of claim 1, wherein updating the instrument representation comprises overlaying or in-painting the instrument representation into the tissue representation based on a current determined position of the high-contrast instrument.

11. The navigational tracking method of claim 1, wherein the position of the high-contrast instrument is determined using pre-segmentation or segmentation of the projection data.

12. The navigational tracking method of claim 1, wherein a target position of the target structure is marked on the tissue representation.

13. The navigational tracking method of claim 12, wherein the target position within the tissue representation is updated based on a registration operation or a manual operation.

14. A navigational imaging system, comprising:
an X-ray source configured to move on a first side of a patient support;
a detector configured to move on a second side of the patient support opposite the first side;
a controller and one or more processing components configured to, alone or in combination:
continuously move the X-ray source and detector along orbital trajectories within a limited angular range and to operate the X-ray source and detector;
acquire a set of projection data at a first frequency or when prompted by a user or physiological monitor;
determine a position of a high-contrast instrument using the set of projection data or images generated using the set of projection data;
update an instrument representation of the high-contrast instrument in a navigational volume at a first rate; and
update a tissue representation depicting low-contrast structures within the navigational volume at a second rate that is equal to or less than the first rate, wherein the low-contrast structures include a target structure or region of the high-contrast instrument.

15. The navigational imaging system of claim 14, wherein the tissue representation of low-contrast structures is updated based upon both the set of projection data and one or more supplemental sets of data acquired using an additional X-ray source and an additional detector.

16. The navigational imaging system of claim 14, wherein the first rate corresponds to a substantially real-time update of the instrument representation within the navigational volume.

17. The navigational imaging system of claim 14, wherein the set of projection data is acquired using a first mode and a second mode having different respective acquisition parameters, wherein the first mode is associated with the step of updating the instrument representation and the second mode is associated with updating the tissue representation.

18. A navigational tracking method comprising:
generating an initial volume comprising a target of a navigational procedure and surrounding tissue;
depicting a representation of a navigational tool within the initial volume;
determining a location of the navigational tool using a subset of projections or images generated from the subset of projections derived from a larger set of projections;
updating, at a first rate, the representation of the navigational tool within the initial volume based on the location;
updating, at a second rate, the depiction of the target and surrounding tissue based on at least the larger set of projections.

19. The navigational tracking method of claim 18, wherein the depiction of the target and surrounding tissue is updated based on the larger set of projections and on an additional set of projections acquired at different view angles relative to the larger set of projections.

20. The navigational tracking method of claim 19, wherein the larger set of projections are acquired while moving the X-ray source in a respective plane above or below a patient.

21. The navigational tracking method of claim 18, wherein the first rate corresponds to a substantially real-time update and the second rate is equal to or slower than the first rate.
